(19) United States
(12) Patent Application Publication
(10) Pub. No.: US 2024/0358537 A1
(43) Pub. Date: Oct. 31, 2024

(54) ORTHOTIC DEVICE FOR MEDICAL BOOT

(71) Applicant: Osborn Medical Corporation, Centennial, CO (US)

(72) Inventors: William D. Davis, Denver, CO (US); Ian P. MacDonald, Denver, CO (US)

(21) Appl. No.: 18/643,367

(22) Filed: Apr. 23, 2024

Related U.S. Application Data
(60) Provisional application No. 63/498,035, filed on Apr. 25, 2023.

(51) Int. Cl. A61F 5/01

(52) U.S. Cl. CPC A61F 5/0111; A61F 5/0195; A61F 2005/0155; A61F 2005/0158

(57) ABSTRACT

An orthotic device for a medical boot includes a first arm for attachment to a first portion of the medical boot, and a second arm for attachment to a second portion of the medical boot. A hinge assembly adjustably fixes an angle between the first and second arms. The hinge assembly includes a first hinge component attached to the first arm, a second hinge component attached to the second arm, and an actuator extending from a proximal end toward a distal end. The distal end of the actuator is biased by a force from a biasing member into a locked position preventing the first and second hinge components from rotating relative to one another. The proximal end of the actuator when actuated, displaces the actuator from the locked position into an unlocked position allowing the second hinge component to rotate relative to the first hinge component.

### CROSS-REFERENCE TO RELATED APPLICATIONS

This application claims priority to U.S. Patent Application No. 63/498,035 filed on Apr. 25, 2023, the disclosure of which is incorporated herein by reference in its entirety.

### BACKGROUND

Patients admitted to a healthcare facility such as a hospital, a nursing home, a rehabilitation center, a long-term care facility, and the like are sometimes bedridden for long periods of time. In some cases, patients experience health problems associated from lying down in one position for too long. For example, it can be desirable to improve blood circulation to prevent clotting, especially for patients who are diagnosed with vascular disease. However, patients who are bedridden for long periods of time can experience vasoconstriction, which is the narrowing of blood vessels causing blood flow to be slowed or blocked.

Also, the feet of bedridden patients should be prevented from pointing downward or leaning to the side, which can lead to foot drop. Foot drop is a condition that may occur after lying in bed for a prolonged period of time without getting up or walking. Foot drop is the dropping of the forefoot due to weakness, damage to the peroneal nerve, or paralysis of the muscles in the anterior portion of the lower leg. It is characterized by the inability or difficulty in moving the ankle and toes upward and thereby leading to the improper rotation of the foot.

Further health problems experienced by patients who are bedridden for prolonged periods of time include skin ulcers that develop as a result of the heel and foot rubbing against the bed sheets and/or pressure being placed on the heel while lying in a hospital bed.

### SUMMARY

In general terms, the present disclosure relates to an orthotic device for a medical boot. In one possible configuration, the orthotic device includes a hinge assembly that adjustably fixes an angle between first and second arms to limit plantar flexion of the foot. Various aspects are described in this disclosure, which include, but are not limited to, the following aspects.

One aspects relates to an orthotic device for a medical boot, the orthotic device comprising: a first arm for attachment to a first portion of the medical boot; a second arm for attachment to a second portion of the medical boot; and a hinge assembly configured to adjustably fix an angle between the first and second arms, the hinge assembly including: a first hinge component attached to the first arm; a second hinge component attached to the second arm; and an actuator extending from a proximal end toward a distal end, the distal end of the actuator being biased by a force from a biasing member into a locked position preventing the first and second hinge components from rotating relative to one another, and the proximal end of the actuator when actuated, displaces the actuator from the locked position into an unlocked position allowing the second hinge component to rotate relative to the first hinge component to adjust the angle between the first and second arms.

Another aspect relates to a kit for mitigating health problems of a bedridden patient, the kit comprising: a medical boot including: a posterior support portion configured to wrap around a lower leg of the bedridden patient; and a planar support portion configured to wrap around a foot of the bedridden patient; and an orthotic device for supporting the planar support portion of the medical boot relative to the posterior support portion of the medical boot, the orthotic device including: a first arm configured for attachment to the posterior support portion of the medical boot; a second arm configured for attachment to the planar support portion of the medical boot; and a hinge assembly configured to adjustably fix an angle between the first and second arms, the hinge assembly including: a first hinge component attached to the first arm; a second hinge component attached to the second arm; and an actuator extending from a proximal end toward a distal end, the distal end of the actuator being biased by a force from a biasing member into a locked position preventing the first and second hinge components from rotating relative to one another, and the proximal end of the actuator when actuated, displaces the actuator from the locked position into an unlocked position allowing the second hinge component to rotate relative to the first hinge component to adjust the angle between the first and second arms.

Another aspect relates to hinge assembly configured to adjustably fix an angle between a first arm and a second arm of an orthotic device, the hinge assembly including: a first hinge component configured for attachment to the first arm; a second hinge component configured for attachment to the second arm; and an actuator extending from a proximal end toward a distal end, the distal end of the actuator being biased by a force from a biasing member into a locked position preventing the first and second hinge components from rotating relative to one another, and the proximal end of the actuator when actuated, displaces the actuator from the locked position into an unlocked position allowing the second hinge component to rotate relative to the first hinge component to adjust the angle between the first and second arms.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a rear isometric view from a right-side perspective of an example of a kit that includes an orthotic device attached to a medical boot.

FIG. 2 is a rear isometric view from a left-side perspective of the kit of FIG. 1.

FIG. 3 is a rear view of the kit of FIG. 1.

FIG. 4 is a front isometric view of an example of the orthotic device of FIG. 1.

FIG. 5 is a front isometric view of the orthotic device of FIG. 1 showing an adjustment of an angle between first and second arms of the orthotic device.

FIG. 6 is a front isometric view of the orthotic device of FIG. 1 showing another adjustment of the angle between first and second arms of the orthotic device.

FIG. 7 is a detailed isometric view of a hinge assembly of the orthotic device of FIG. 1 that pivotally connects the first and second arms.

FIG. 8 is an exploded isometric view of the hinge assembly of FIG. 7.

FIG. 9A is a cross-sectional view of the hinge assembly of FIG. 7, the hinge assembly being shown in a locked position.

FIG. 9B is a cross-sectional view of the hinge assembly of FIG. 7, the hinge assembly being shown in an unlocked position.

FIG. 10 schematically illustrates an example of a method of operating the hinge assembly of FIG. 7 to adjust the angle between first and second arms of the orthotic device.

FIG. 11 is a side view of the hinge assembly of FIG. 7 when adjusted to provide an acute angle between the first and second arms of the orthotic device.

FIG. 12 is a side view of the hinge assembly of FIG. 7 when adjusted to provide a right angle between the first and second arms of the orthotic device.

FIG. 13 is a side view of the hinge assembly of FIG. 7 when adjusted to provide an obtuse angle between the first and second arms of the orthotic device.

FIG. 14 illustrates an isometric view of another example of an orthotic device that can be used to support the medical boot of FIG. 1.

FIG. 15 is an isometric exploded view of the orthotic device of FIG. 14.

FIG. 16 is an isometric exploded view of the orthotic device of FIG. 14.

FIG. 17 is an isometric cross-sectional view of the orthotic device of FIG. 14.

FIG. 18 shows an angle between first and second arms of the orthotic device of FIG. 14 adjusted to be 0 degrees.

FIG. 19 shows the angle between first and second arms of the orthotic device of FIG. 14 adjusted to be 180 degrees.

### DETAILED DESCRIPTION

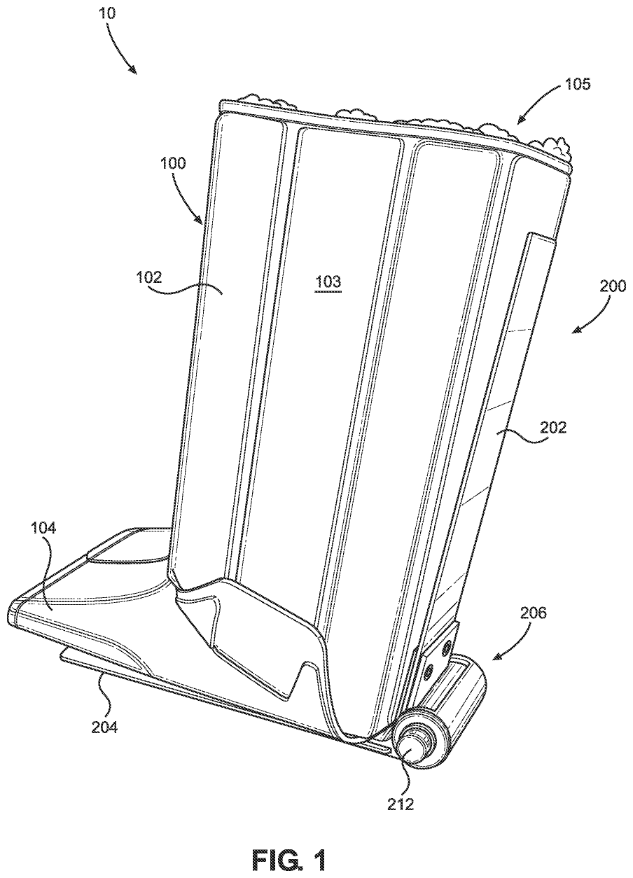

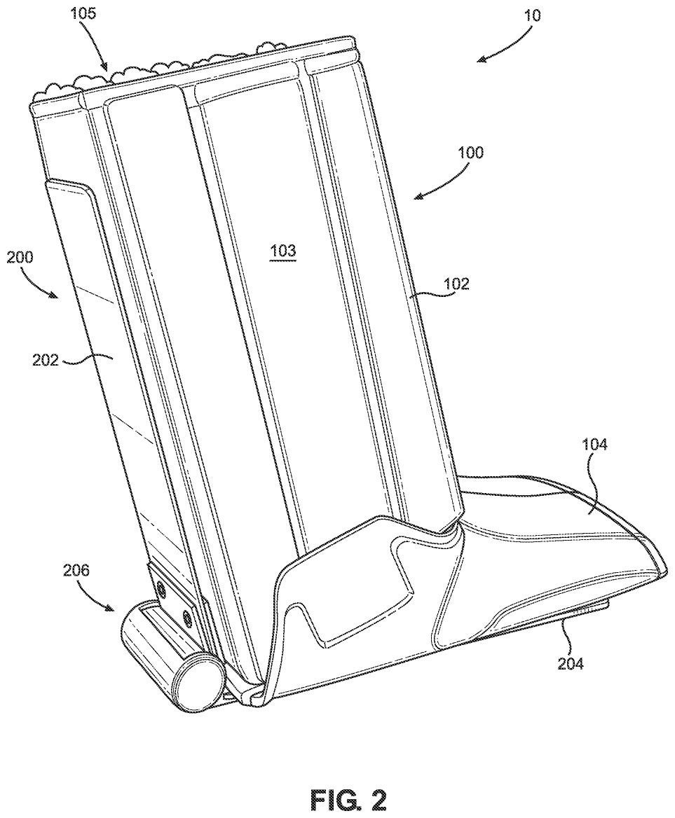

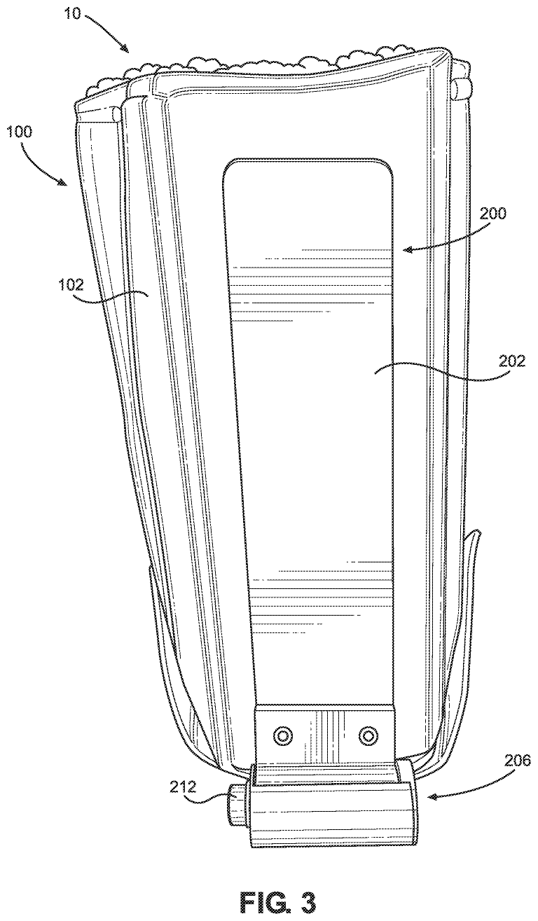

FIG. 1 is a rear isometric view from a right-side perspective of an example of a kit 10 for mitigating health problems of a bedridden patient. FIG. 2 is a rear isometric view from a left-side perspective of the kit 10. FIG. 3 is a rear view of the kit 10. As shown in FIGS. 1-3, the kit 10 includes an orthotic device 200 attached to a medical boot 100. As will be described in more detail, the orthotic device 200 includes a hinge assembly 206 that is operable to adjustably limit plantar flexion of the foot when the orthotic device 200 is attached to the medical boot 100. The orthotic device 200 helps to prevent foot drop by supporting the foot in a preferred position such as by keeping the foot in an upright position. This prevents the foot from pointing toward the foot of the bed or falling over to one side while the patient remains lying down.

The medical boot 100 can be worn by a patient admitted to a healthcare facility such as a hospital, a nursing home, a rehabilitation center, a long-term care facility, and the like. The medical boot 100 can also be worn by a person who is bedridden in their own home.

The medical boot 100 provides support for the foot by off-loading weight from the heel which can help protect against pressure ulcers formed on the skin. For example, the medical boot 100 can be used to float the heel over an empty space inside the boot. The medical boot 100 can also be used to keep the patient's foot warm to improve blood circulation.

As shown in FIGS. 1-3, the medical boot 100 includes a posterior support portion 102 that is configured to wrap around a lower leg of the bedridden patient. The medical boot 100 also includes a planar support portion 104 that extends from the posterior support portion 102. The planar support portion is configured to wrap around a foot of the bedridden patient.

In some examples, the posterior support portion 102 and the planar support portion 104 are integrated together into a single component of the medical boot 100. In other examples, the posterior support portion 102 and the planar support portion 104 are separate components that can removably attach together using one or more fasteners such as hook-and-loop fasteners.

The posterior support portion 102 covers a part of the lower leg of the patient including at least a portion of the calf and ankle. The posterior support portion 102 includes one or more foam pieces sandwiched between an exterior fabric layer 103 and an interior fabric layer 105. The exterior fabric layer 103 can be made of loop material that can attach to a hook material of a hook-and-loop fastener. The interior fabric layer 105 can include soft, insulating fabric such as fleece. The one or more foam pieces and interior fabric layer provide cushioning, protect against pressure ulcers, and maintain the patient's leg warm to improve blood flow circulation.

The planar support portion 104 of the medical boot 100 extends from the posterior support portion 102 and covers at least a part of the foot area. The planar support portion 104 helps protect against pressure ulcers formed on the skin and keeps the foot warm. In some examples, the planar support portion 104 is hinged with the posterior support portion 102.

The orthotic device 200 includes a first arm 202 that attaches to the posterior support portion 102 of the medical boot 100. The orthotic device 200 includes a second arm 204 that attaches to the planar support portion 104 of the medical boot 100. The orthotic device 200 further includes the hinge assembly 206 that can be used to adjustably fix an angle between the first and second arms 202, 204 to control plantar flexion and dorsiflexion of the planar support portion 104 relative to the posterior support portion 102 of the medical boot. The hinge assembly 206 includes an actuator 212 that can be actuated by a user of the kit 10 such as a caregiver to adjust the angle between the first and second arms 202, 204 of the orthotic device.

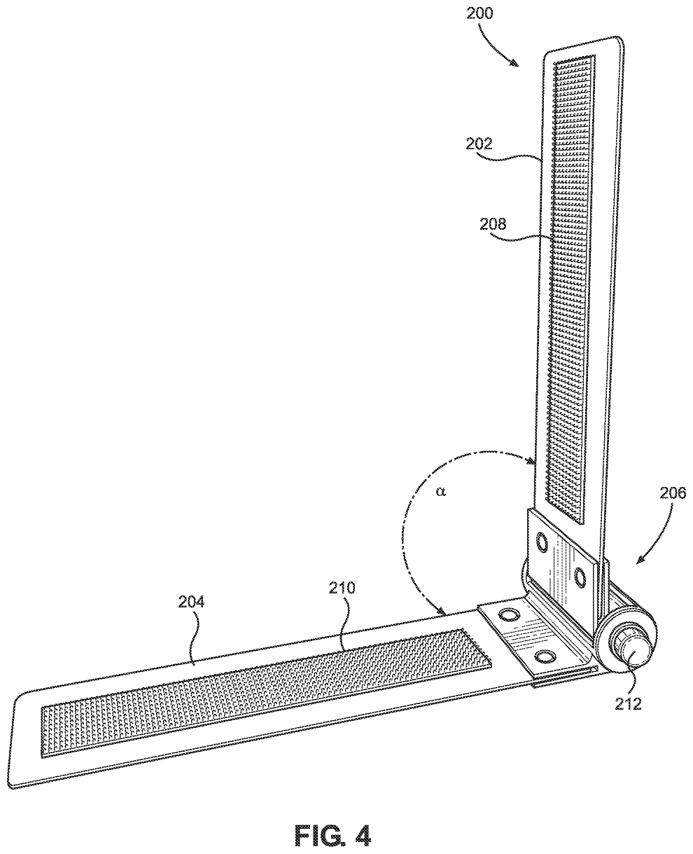

FIG. 4 is a front isometric view of an example of the orthotic device 200 separated from the medical boot 100. In this example, the first arm 202 includes a first fastener 208 for removably attaching the first arm 202 to the posterior support portion 102 of the medical boot. The second arm 204 includes a second fastener 210 for removably attaching the second arm 204 to the planar support portion 104 of the medical boot. In this example, the first and second fasteners 208, 210 each include strips of hook material that removably attach to the loop material on the exterior fabric layer 103 for attaching the orthotic device 200 to the medical boot 100.

In further examples, other types of fasteners such as snap fasteners, also called snap buttons, can be used to removably attach the orthotic device 200 to the medical boot 100. Also, alternative means for removably attaching the orthotic device 200 to the medical boot 100 are contemplated. For example, in some examples, the posterior support portion 102 can include a sleeve or pocket into which the first arm 202 can slide into and the planar support portion 104 can also include a sleeve or pocket into which the second arm 204 can slide into to removably attach the orthotic device 200 to the medical boot 100.

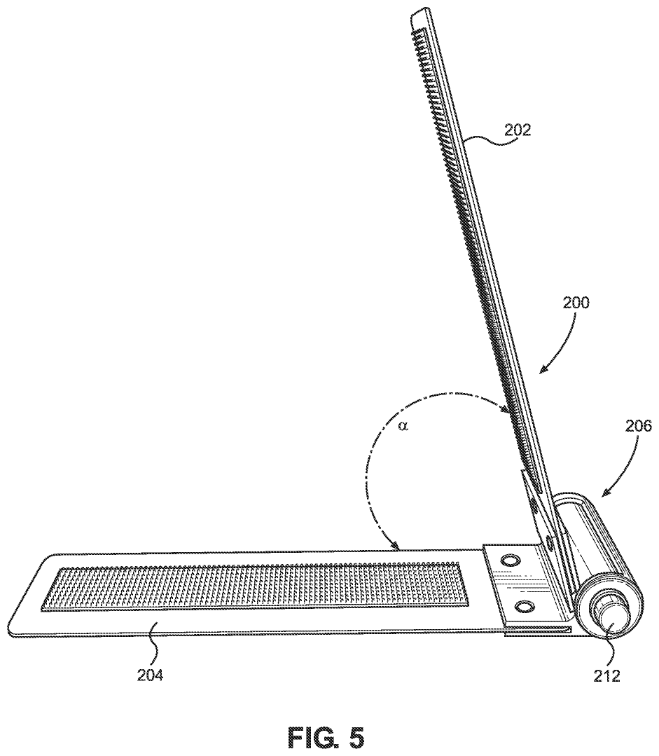

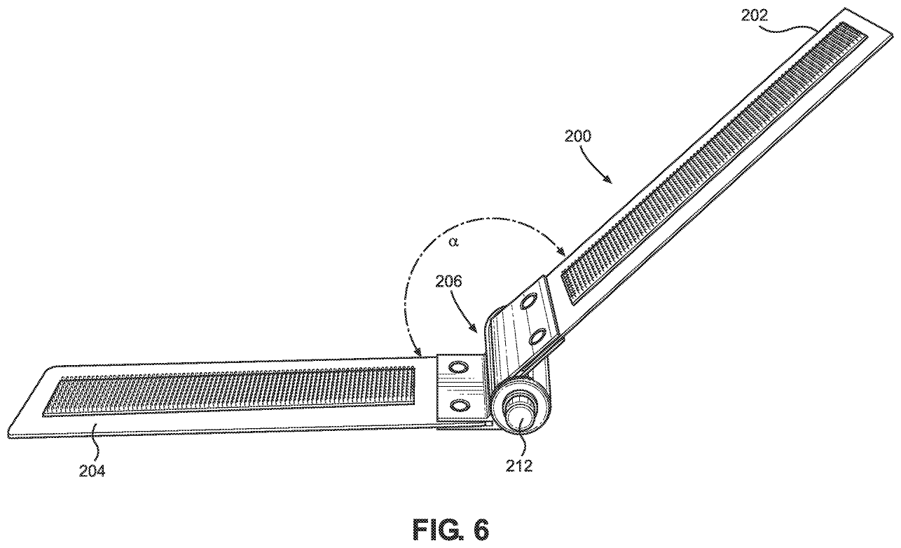

FIG. 5 is a front isometric view of the orthotic device 200 showing an adjustment of the angle α between first and second arms 202, 204 of the orthotic device. FIG. 6 is another front isometric view of the orthotic device 200 showing another adjustment of the angle α between first and second arms 202, 204 of the orthotic device. Referring now to FIGS. 4-6, the hinge assembly 206 can be operated by a user of the orthotic device 200 to fixedly adjust the angle α between the first and second arms 202, 204 of the orthotic device. The hinge assembly adjustably fixes the angle α between the first and second arms 202, 204 in a range between an acute angle (i.e., an angle less than 90 degrees) and an obtuse angle (i.e., an angle greater than 90 degrees).

FIG. 4 illustrates an example where the hinge assembly 206 is used to fix a right angle (90 degrees) between the first and second arms 202, 204. FIG. 5 illustrates an example where the hinge assembly 206 is used to fix an acute angle (less than 90 degrees) between the first and second arms 202, 204. FIG. 6 illustrates an example where the hinge assembly 206 is used to fix an obtuse angle (greater 90 degrees) between the first and second arms 202, 204.

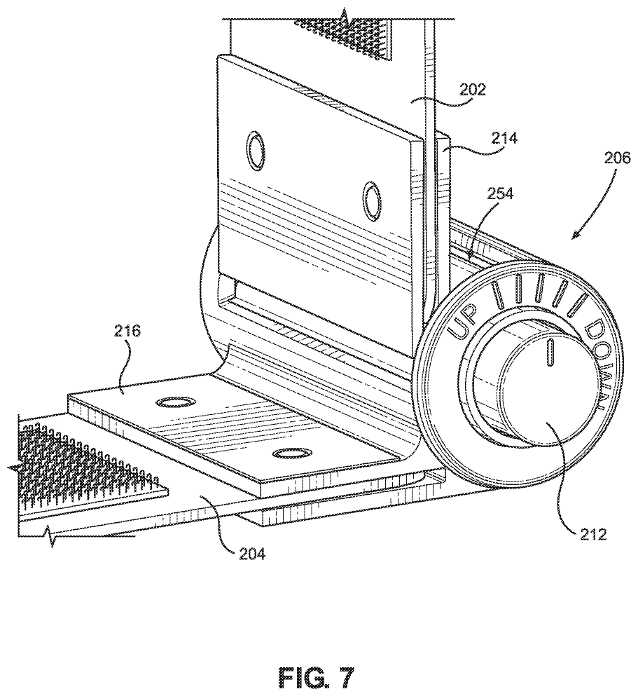

FIG. 7 is a detailed isometric view of the hinge assembly 206 that pivotally connects the first and second arms 202, 204. As shown in FIG. 7, the hinge assembly 206 includes a first hinge component 214 associated with the first arm 202, and a second hinge component 216 associated with the second arm 204. For example, the first hinge component 214 is configured to move the first arm 202 relative to the second arm 204, and the second hinge component 216 is configured to move the second arm 204 relative to the first arm 202.

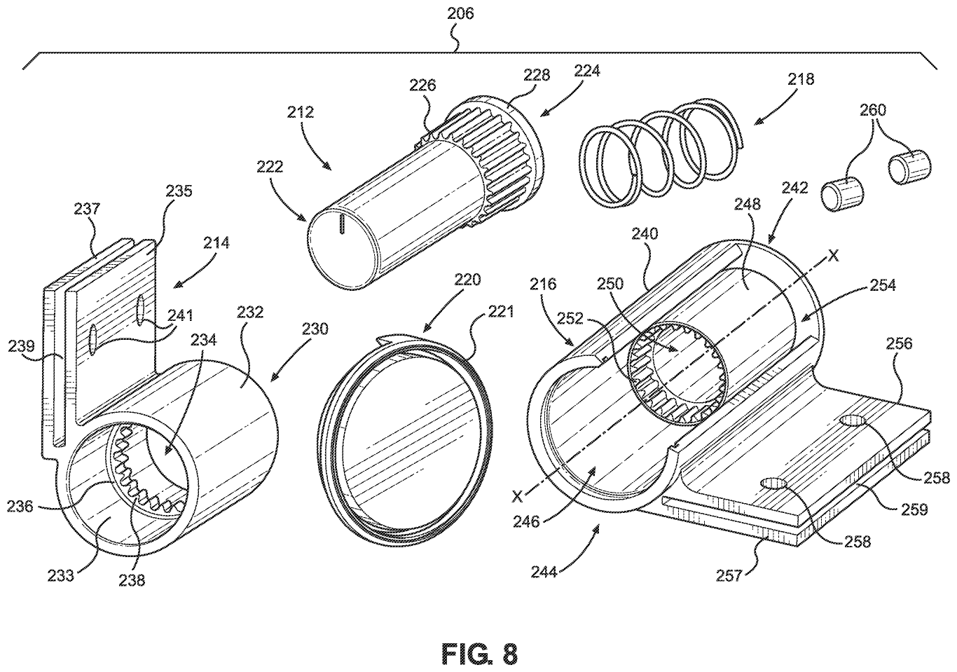

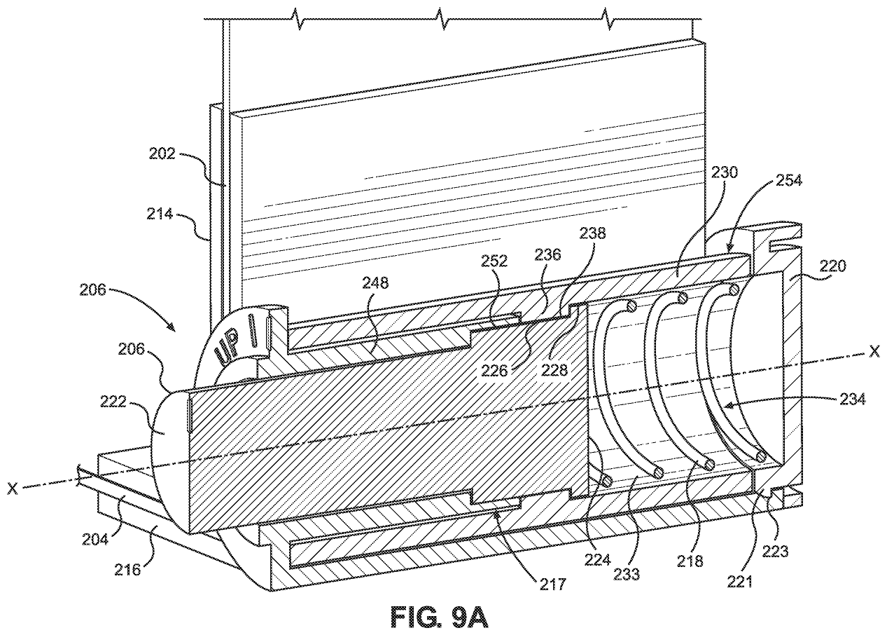

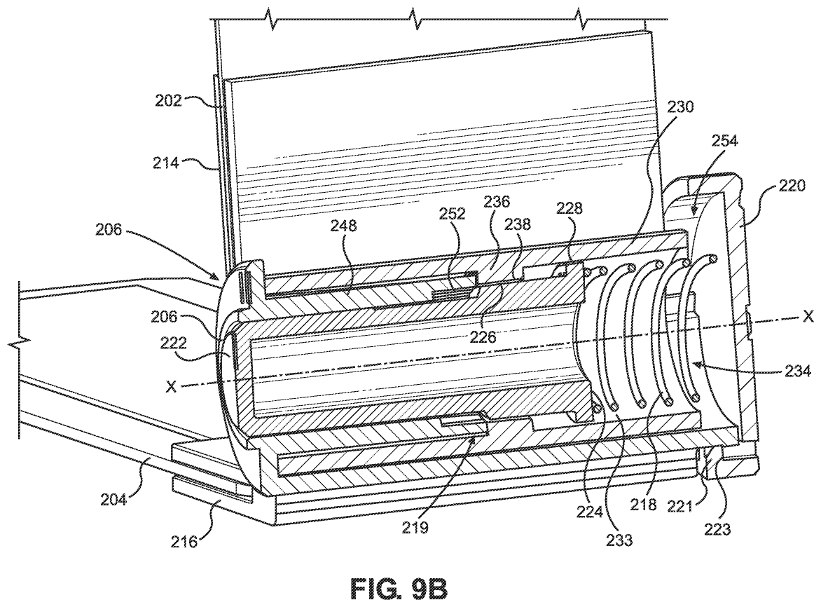

FIG. 8 is an exploded isometric view of the hinge assembly 206. FIG. 9A is a cross-sectional view of the hinge assembly 206 shown in a locked position 217. FIG. 9B is a cross-sectional view of the hinge assembly 206 shown in an unlocked position 219. The hinge assembly 206 includes an actuator 212 that extends from a proximal end 222 to a distal end 224. The distal end 224 is biased by a biasing member 218 into a locked position 217 that prevents the first and second hinge components 214, 216 from pivoting relative to each other. The proximal end 222 of the actuator 212 when actuated, displaces the actuator 212 from the locked position 217 to an unlocked position 219 that allows the second hinge component 216 to pivot relative to the first hinge component 214 to adjust the angle α between the first and second arms 202, 204.

In the example shown in FIGS. 7-9, actuation of the actuator 212 includes pushing the proximal end 222 of the actuator 212 causing the biasing member 218 to compress between a housing 240 of the second hinge component 216 and the distal end 224 of the actuator 212. In alternative examples, it is contemplated that actuation of the actuator 212 can include pulling the proximal end 222 of the actuator 212 causing the biasing member 218 to expand between the housing 240 of the second hinge component 216 and the distal end 224 of the actuator 212.

As shown in FIGS. 8 and 9, the first hinge component 214 includes a tubular body 230 having an exterior surface 232 and an interior surface 233. The tubular body 230 defines a first internal cavity 234 that includes a protrusion 236 projecting from the interior surface 233. The protrusion 236 includes a first plurality of teeth 238 (also known as cogs).

The second hinge component 216 includes the housing 240 that extends between a proximal end 242 and a distal end 244. In the example shown in FIGS. 8 and 9, an end cap 220 can attach to the distal end 244 of the housing 240. The end cap 220 includes a first interlocking component 221 such as a tab that can be pushed into a second interlocking component 223 on the housing 240 (see FIG. 9A, 9B) such as a lip providing a snap-fit connection. Alternatively, the end cap 220 can be screwed onto the distal end 244 of the housing 240, or can be attached to the housing 240 of the second hinge component 216 by other means. The housing 240 defines a second internal cavity 246 for receiving the tubular body 230 of the first hinge component 214.

The housing 240 includes an internal body 248 that extends from the proximal end 242 into the second internal cavity 246. The internal body 248 is received inside the first internal cavity 234 of the first hinge component 214. The internal body 248 defines a third internal cavity 250 having a second plurality of teeth 252 (also known as cogs). As shown in FIG. 9, the second plurality of teeth 252 align with the first plurality of teeth 238 on the protrusion 236 that projects from the interior surface 233 of the first hinge component 214.

The biasing member 218 is disposed inside the first internal cavity 234 in the tubular body 230 of the first hinge component 214. In the example shown in FIG. 9, the biasing member 218 is compressed between the distal end 224 of the actuator 212 and the end cap 220 attached to the distal end 244 of the housing 240 of the second hinge component 216. In some examples, the biasing member 218 is a coil spring. In some further examples, the coil spring is a compression coil spring. As shown in FIGS. 8 and 9, the distal end 224 of the actuator 212 includes a flange 228 that increases a surface area of the distal end 224 in contact with the biasing member 218.

As shown in FIG. 9A, the distal end 224 of the actuator 212 includes a third plurality of teeth 226 (also known as cogs) that engage the first and second pluralities of teeth 238, 252 when the actuator 212 is in the locked position 217. The locked position 217 prevents the first and second hinge components 214, 216 from pivoting relative to one another because the third plurality of teeth 226 on the actuator 212 prevent relative rotation between the first and second pluralities of teeth 238, 252 of the first and second hinge components 214, 216.

As shown in FIG. 9B, when in the unlocked position 219, the third plurality of teeth 226 of the actuator 212 disengage at least one of the first and second pluralities of teeth 238, 252 allowing the first and second hinge components 214, 216 to rotate relative to each other.

As shown in FIG. 9, actuation of the actuator 212 includes pushing the proximal end 222 of the actuator 212 causing the biasing member 218 to compress between the distal end 224 of the actuator 212 and the end cap 220 attached to the distal end 244 of the housing 240 of the second hinge component 216. This causes the third plurality of teeth 226 of the actuator 212 to disengage the second plurality of teeth 252 of the second hinge component 216 allowing the second hinge component 216 to rotate relative to the first hinge component 214. This allows adjustment of the angle α between the first and second arms 202, 204 of the orthotic device 200.

When the actuator 212 is released (i.e., the proximal end 222 of the actuator 212 is no longer being pushed), a spring force of the biasing member 218 that resists compression pushes against the distal end 224 of the actuator 212, which causes the actuator 212 to return to the locked position 217. As shown in FIG. 9, the flange 228 of the actuator 212 abuts the protrusion 236 of the tubular body 230 which prevents further displacement of the actuator 212 by the spring force of the biasing member 218. When the actuator returns to the locked position 217, the third plurality of teeth 226 of the actuator 212 engage the first and second pluralities of teeth 238, 252, which prevents rotation of the first and second hinge components 214, 216. In this manner, the actuator can be pushed by a user to allow adjustment of the angle α between the first and second arms 202, 204, and can thereafter be released by the user to fix the angle α between the first and second arms 202, 204 as desired by the user of the orthotic device 200.

In alternative examples, actuation of the actuator 212 can include pulling the proximal end 222 of the actuator to cause the third plurality of teeth 226 of the actuator 212 disengage at least one of the first and second pluralities of teeth 238, 252 allowing the first and second hinge components 214, 216 to rotate relative to each other. In such examples, the biasing member 218 includes a tension coil spring attached to the distal end 224 of the actuator 212 that includes a spring force that resists stretching such that when the proximal end 222 of the actuator is released (i.e., the proximal end 222 of the actuator 212 is no longer being pulled), the spring force of the biasing member 218 pulls the distal end 224 of the actuator 212, which causes the actuator 212 to return to the locked position 217 such that the third plurality of teeth 226 of the actuator 212 engage the first and second pluralities of teeth 238, 252, which prevents rotation of the first and second hinge components 214, 216. Additional means for actuating the actuator 212 to transition between the locked position 217 and the unlocked position 219 are contemplated.

As shown in FIGS. 7-9, the first hinge component 214 includes a slot 239 between first and second plates 235, 237. The first and second plates 235, 237 include bore holes 241 such that when the first arm 202 is positioned inside the slot 239, a fastener 260 such as a rivet, bolt, screw, and the like can be used to attach the first arm 202 to the first hinge component 214. Additional means for attaching the first arm 202 to the first hinge component 214 are possible. In alternative examples, the first arm 202 and the first hinge component 214 are formed from a single piece of material such that they are an integral, continuous component.

Similarly, the second hinge component 216 includes a slot 259 between first and second plates 256, 257. The first and second plates 256, 257 include bore holes 258 such that when the second arm 204 is positioned inside the slot 259, a fastener 260 such as a rivet, bolt, screw, and the like can be used to attach the second arm 204 to the second hinge component 216. Additional means for attaching the second arm 204 to the second hinge component 216 are possible. In alternative examples, the second arm 204 and the second hinge component 216 are formed from a single piece of material such that they are an integral, continuous component.

As further shown in FIG. 7-9, the housing 240 of the second hinge component 216 can include a window 254 that at least partially exposes the tubular body 230 of the first hinge component 214 received inside the second internal cavity 246. As shown in FIG. 9, the tubular body 230 surrounds the internal body 248 of the second hinge component 216. The third internal cavity 250 of the housing 240 defines a central axis X-X about which the tubular body 230 is rotatable when the actuator 212 is in the unlocked position 219. The window 254 allows a user of the orthotic device 200 to view rotation of the tubular body 230 about the central axis X-X.

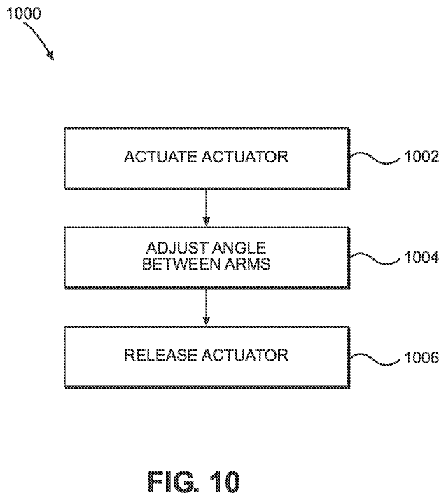

FIG. 10 schematically illustrates an example of a method 1000 of operating the hinge assembly 206 to adjust the angle α between first and second arms 202, 204 of the orthotic device 200. The method 1000 includes an operation 1002 of actuating the actuator 212. In accordance with the examples shown in FIGS. 7-9, operation 1002 includes pushing the proximal end 222 of the actuator 212 such that the third plurality of teeth 226 of the actuator 212 disengage at least one of the first and second pluralities of teeth 238, 252 allowing the first and second hinge components 214, 216 to rotate relative to each other. In alternative examples, operation 1002 can include pulling the proximal end 222 of the actuator 212 such that the third plurality of teeth 226 of the actuator 212 disengage at least one of the first and second pluralities of teeth 238, 252.

Next, the method 1000 includes an operation 1004 of adjusting the angle α between the first and second arms 202, 204. For example, operation 1004 can include adjusting the angle α between the first and second arms 202, 204 to have an acute angle (i.e., less than 90 degrees).

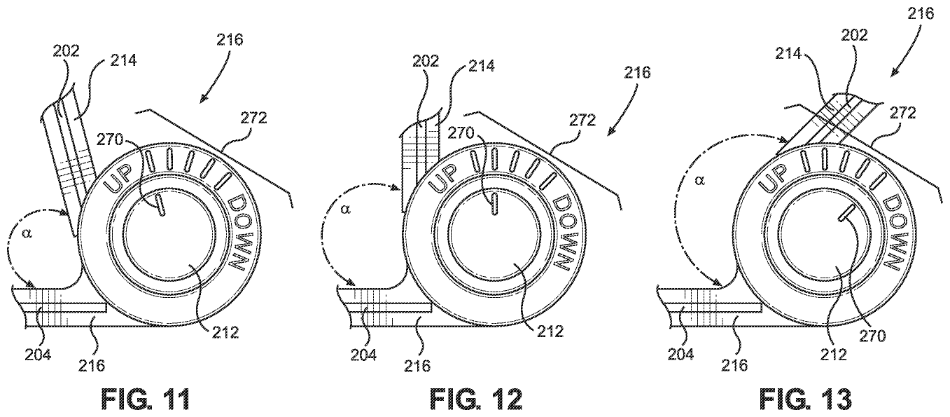

FIG. 11 is a side view of the hinge assembly 206 when adjusted to provide an acute angle between the first and second arms 202, 204 of the orthotic device 200. As shown in FIG. 11, the proximal end 222 of the actuator 212 can include a dial 270 that aligns with one or more markings 272 depicted on the proximal end 242 of the housing 240 of the second hinge component 216. The markings 272 represent stops between the minimum and maximum angles allowed for separating the first and second arms 202, 204 of the orthotic device 200. From left to right, the stops gradually increase the angle α between the first and second arms 202, 204. The dial 270 and the markings 272 allow a user to view an amount or degree of angular separation that is adjusted between the first and second arms 202, 204 of the orthotic device 200.

Operation 1004 can alternatively include adjusting the angle α between the first and second arms 202, 204 to have a right angle (i.e., 90 degrees). FIG. 12, which is another side view of the hinge assembly 206, shows the proximal ends of the actuator 212 and the housing 240 of the second hinge component 216 when the hinge assembly 206 is adjusted to provide a right angle between the first and second arms 202, 204 of the orthotic device 200.

Operation 1004 can alternatively include adjusting the angle α between the first and second arms 202, 204 to have an obtuse angle (i.e., greater than 90 degrees). FIG. 13, which is another side view of the hinge assembly 206, shows the proximal ends of the actuator 212 and the housing 240 of the second hinge component 216 when the hinge assembly 206 is adjusted to provide an obtuse angle between the first and second arms 202, 204 of the orthotic device 200.

As further shown in FIG. 10, the method 1000 includes an operation 1006 of releasing the actuator 212. In accordance with the examples shown in FIGS. 7-9, operation 1006 includes stop pushing the proximal end 222 of the actuator 212 such that the spring force of the biasing member 218 pushes against the distal end 224 of the actuator 212, which causes the actuator 212 to return to the locked position 217. Alternatively, operation 1006 can include stop pulling the proximal end 222 of the actuator 212 such that the spring force of the biasing member 218 pulls the distal end 224 of the actuator 212, which causes the actuator 212 to return to the locked position 217. When the actuator returns to the locked position 217, the third plurality of teeth 226 of the actuator 212 engage the first and second pluralities of teeth 238, 252, which prevents rotation of the first and second hinge components 214, 216 such that the angle α between the first and second arms 202, 204 is fixed.

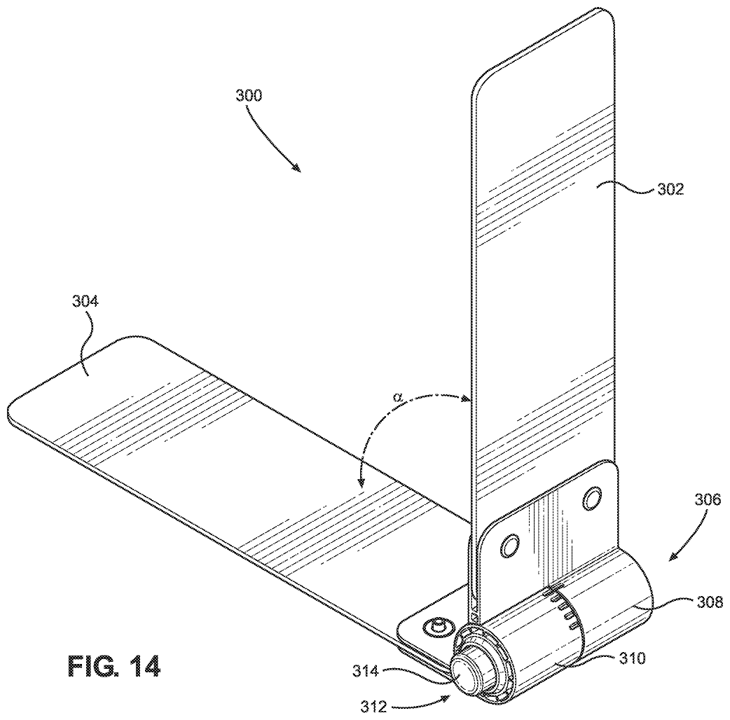

FIG. 14 illustrates an isometric view of another example of an orthotic device 300 that can be used to support the medical boot 100. The orthotic device 300 similarly includes a first arm 302 for attachment to the posterior support portion 102 of the medical boot 100, and a second arm 304 for attachment to the planar support portion 104 of the medical boot 100. The orthotic device 300 further includes a hinge assembly 306 configured to adjustably fix an angle α between the first and second arms 302, 304 of the orthotic device 300.

Like in the examples described above, the first arm 302 can include a first fastener to removably attach the first arm 302 to the posterior support portion 102 of the medical boot 100, and the second arm 304 can include a second fastener to removably attach the second arm 304 to the planar support portion 104 of the medical boot 100. The first and second fasteners can include strips of hook material that removably attach to loop material on the medical boot 100.

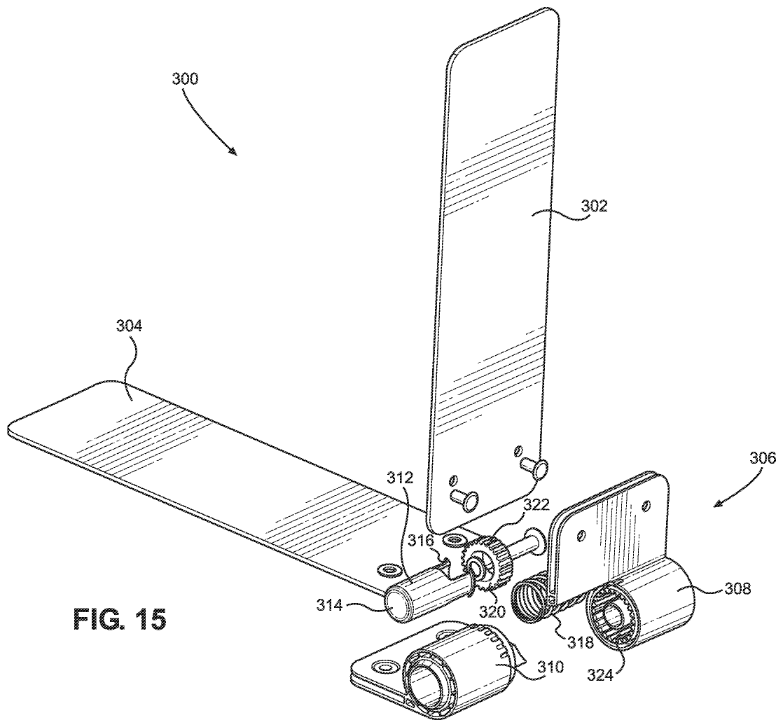

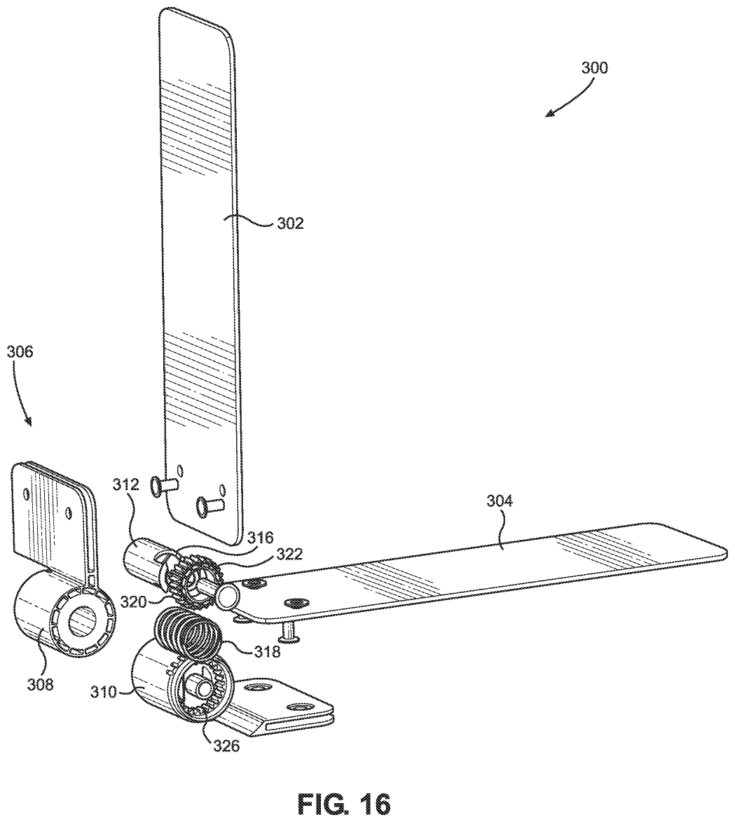

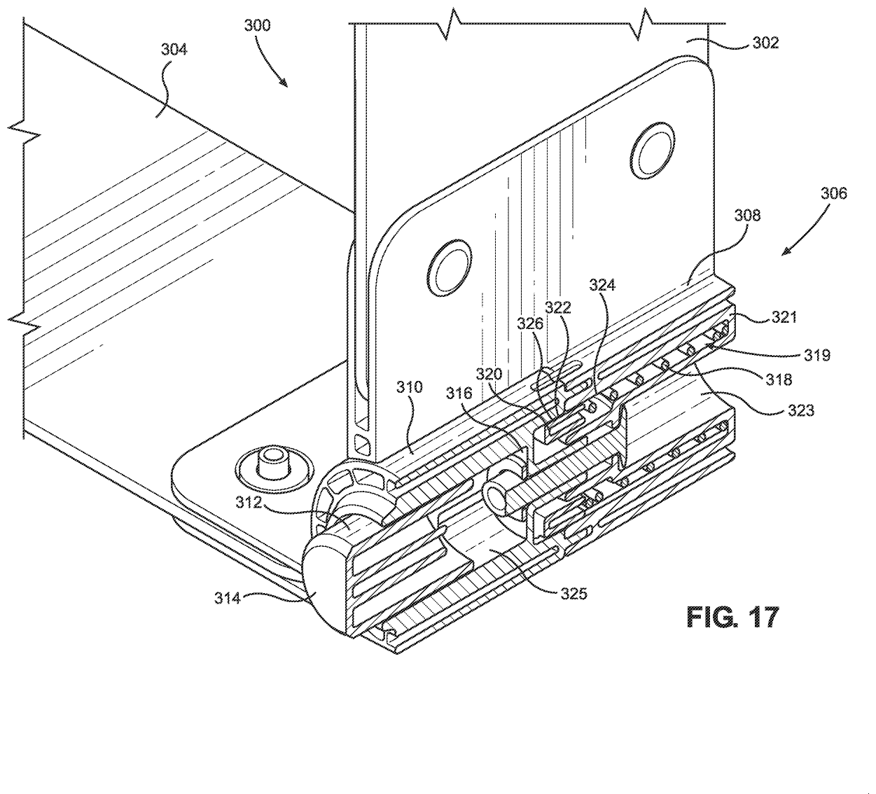

FIGS. 15 and 16 are isometric exploded views of the orthotic device 300. FIG. 17 is an isometric cross-sectional view of the orthotic device 300. Referring now to FIGS. 14-17, the hinge assembly 306 includes a first hinge component 308 for attachment to the first arm 302, and a second hinge component 310 for attachment to the second arm 304.

The hinge assembly 306 includes an actuator 312 extends from a proximal end 314 toward a distal end 316. A biasing member 318 biases a gear 320 to engage the distal end 316 of the actuator 312. In some examples, the biasing member 318 is a spring.

As shown in FIG. 17, the biasing member 318 is disposed inside an internal cavity 319 of the first hinge component 308. The biasing member 318 is compressed between the gear 320 and a distal end 321 of the internal cavity 319 of the first hinge component 308.

The biasing member 318 causes the hinge assembly 306 to be in a locked position when the actuator 312 is not engaged by a user. When in the locked position, the hinge assembly 306 prevents the first and second hinge components 308, 310 from rotating relative to one another such that the angle α between the first and second arms 302, 304 is fixed.

The gear 320 has a first set of teeth 322 that engage a second set of teeth 324 on an interior surface 323 of the first hinge component 308 and a third set of teeth 326 on an interior surface 325 of the second hinge component 310. When in the locked position, the first set of teeth 322 on the gear 320 engage both the second and third sets of teeth 324, 326 such that the first and second hinge components 308, 310 are prevented from rotating relative to one another, which causes the angle α between the first and second arms 302, 304 to be fixed.

When the actuator 312 is engaged by the user pushing on the proximal end 314, the distal end 316 of the actuator 312 displaces the gear 320 such that the first set of teeth 322 on the gear 320 disengage the third set of teeth 326 of the second hinge component 310 and the biasing member 318 compresses. This causes the hinge assembly 306 to be in an unlocked position. When in the unlocked position, the first and second arms 302, 304 are able to rotate relative to one another. When in the unlocked position, the angle α between the first and second arms 302, 304 is adjustable anywhere between 0 degrees and 180 degrees. When the proximal end 314 of the actuator 312 is released, a biasing force of the biasing member 318 returns the actuator 312 from the unlocked position to the locked position.

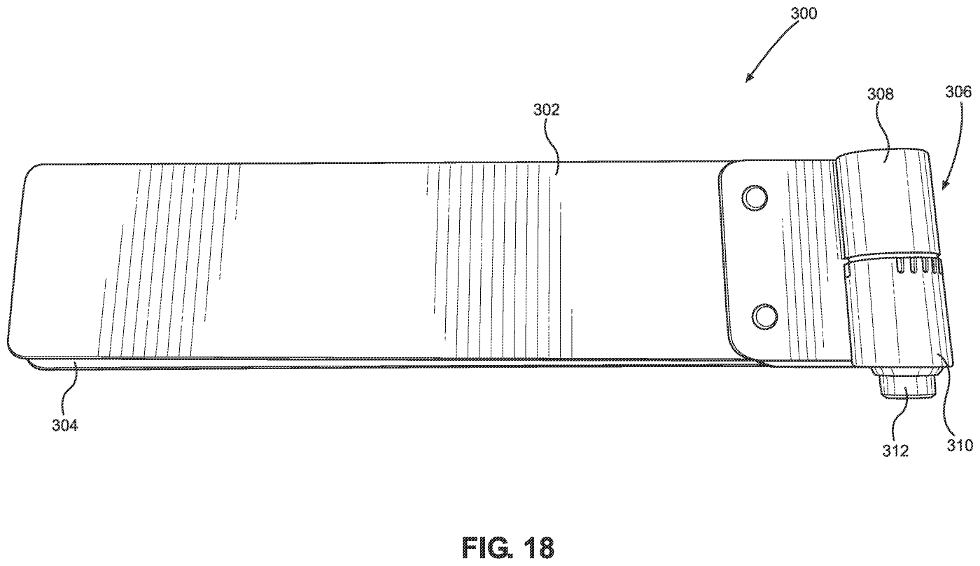

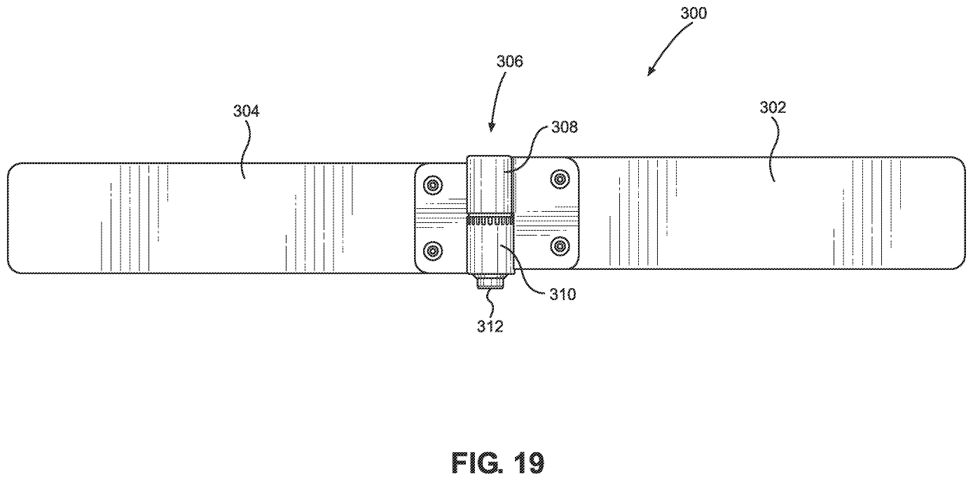

FIG. 18 shows the angle α between the first and second arms 302, 304 of the orthotic device 300 adjusted to be 0 degrees. FIG. 19 shows the angle α between the first and second arms 302, 304 of the orthotic device 300 adjusted to be 180 degrees. Advantageously, when the angle α between the first and second arms 302, 304 is adjusted to be 0 degrees such that the first and second arms 302, 304 are facing and parallel with one another (see FIG. 18), the size of the orthotic device 300 is compact, which makes it easier for shipping the orthotic device 300.

The various embodiments described above are provided by way of illustration only and should not be construed to be limiting in any way. Various modifications can be made to the embodiments described above without departing from the true spirit and scope of the disclosure.

What is claimed is:

1. An orthotic device for a medical boot, the orthotic device comprising:
a first arm for attachment to a first portion of the medical boot;
a second arm for attachment to a second portion of the medical boot; and
a hinge assembly configured to adjustably fix an angle between the first and second arms, the hinge assembly including:
a first hinge component attached to the first arm;
a second hinge component attached to the second arm; and
an actuator extending from a proximal end toward a distal end, the distal end of the actuator being biased by a force from a biasing member into a locked position preventing the first and second hinge components from rotating relative to one another, and the proximal end of the actuator when actuated, displaces the actuator from the locked position into an unlocked position allowing the second hinge component to rotate relative to the first hinge component to adjust the angle between the first and second arms.

2. The orthotic device of claim 1, further comprising:
a gear engaging the distal end of the actuator, the gear having a first set of teeth;
a second set of teeth on an interior surface of the first hinge component; and
a third set of teeth on an interior surface of the second hinge component, wherein when in the locked position, the first set of teeth engages both the second and third sets of teeth preventing the first and second hinge components to rotate relative to one another.

3. The orthotic device of claim 2, wherein displacing the actuator from the locked position into the unlocked position causes the first set of teeth on the gear to disengage the third set of teeth allowing the first and second hinge components to rotate relative to one another.

4. The orthotic device of claim 3, wherein the biasing member causes displacement of the actuator from the unlocked position into the locked position when the proximal end of the actuator is released.

5. The orthotic device of claim 2, wherein the biasing member is disposed inside an internal cavity of the first hinge component.

6. The orthotic device of claim 5, wherein the biasing member is compressed between the gear and a distal end of the internal cavity of the first hinge component.

7. The orthotic device of claim 1, wherein the first arm includes a first fastener to removably attach the first arm to the first portion of the medical boot, and the second arm includes a second fastener to removably attach the second arm to the second portion of the medical boot.

8. The orthotic device of claim 7, wherein the first and second fasteners include strips of hook material that removably attach to loop material on the medical boot.

9. The orthotic device of claim 1, wherein the hinge assembly adjustably fixes the angle between the first and second arms in a range between 0 degrees and 180 degrees.

10. A kit for mitigating health problems of a bedridden patient, the kit comprising:
a medical boot including:
a posterior support portion configured to wrap around a lower leg of the bedridden patient; and
a planar support portion configured to wrap around a foot of the bedridden patient; and
an orthotic device for supporting the planar support portion of the medical boot relative to the posterior support portion of the medical boot, the orthotic device including:
a first arm configured for attachment to the posterior support portion of the medical boot;
a second arm configured for attachment to the planar support portion of the medical boot; and
a hinge assembly configured to adjustably fix an angle between the first and second arms, the hinge assembly including:
a first hinge component attached to the first arm;
a second hinge component attached to the second arm; and
an actuator extending from a proximal end toward a distal end, the distal end of the actuator being biased by a force from a biasing member into a locked position preventing the first and second hinge components from rotating relative to one another, and the proximal end of the actuator when actuated, displaces the actuator from the locked position into an unlocked position allowing the second hinge component to rotate relative to the first hinge component to adjust the angle between the first and second arms.

11. The kit of claim 10, wherein the first arm includes a first fastener for securing the first arm to the posterior support portion of the medical boot, and the second arm includes a second fastener for securing the second arm to the planar support portion of the medical boot.

12. The kit of claim 11, wherein the first and second fasteners include strips of hook material that removably attach to loop material on the medical boot.

13. The kit of claim 10, wherein the hinge assembly further includes:
a gear engaging the distal end of the actuator, the gear having a first set of teeth;
a second set of teeth on an interior surface of the first hinge component; and
a third set of teeth on an interior surface of the second hinge component, wherein when in the locked position, the first set of teeth engages both the second and third sets of teeth preventing the first and second hinge components to rotate relative to one another.

14. The kit of claim 13, wherein displacing the actuator from the locked position into the unlocked position causes the first set of teeth on the gear to disengage the third set of teeth allowing the first and second hinge components to rotate relative to one another.

15. The kit of claim 13, wherein the biasing member is compressed between the gear and a distal end of an internal cavity of the first hinge component.

16. The kit of claim 10, wherein the biasing member causes displacement of the actuator from the unlocked position into the locked position when the proximal end of the actuator is released.

17. The kit of claim 10, wherein the hinge assembly adjustably fixes the angle between the first and second arms in a range between 0 degrees and 180 degrees.

18. A hinge assembly configured to adjustably fix an angle between a first arm and a second arm of an orthotic device, the hinge assembly including:
a first hinge component configured for attachment to the first arm;
a second hinge component configured for attachment to the second arm; and
an actuator extending from a proximal end toward a distal end, the distal end of the actuator being biased by a force from a biasing member into a locked position preventing the first and second hinge components from rotating relative to one another, and the proximal end of the actuator when actuated, displaces the actuator from the locked position into an unlocked position allowing the second hinge component to rotate relative to the first hinge component to adjust the angle between the first and second arms.

19. The hinge assembly of claim 18, further comprising:
a gear engaging the distal end of the actuator, the gear having a first set of teeth;
a second set of teeth on an interior surface of the first hinge component; and
a third set of teeth on an interior surface of the second hinge component, wherein when in the locked position, the first set of teeth engages both the second and third sets of teeth preventing the first and second hinge components to rotate relative to one another.

20. The hinge assembly of claim 19, wherein displacing the actuator from the locked position into the unlocked position causes the first set of teeth on the gear to disengage the third set of teeth allowing the first and second hinge components to rotate relative to one another.
